(19) United States
(12) Patent Application Publication
(10) Pub. No.: US 2011/0256221 A1
(43) Pub. Date: Oct. 20, 2011

(54) PHARMACEUTICAL FORMULATIONS OF OLMESARTAN

(76) Inventors: Farhad Farshi, Istanbul (TR); Recep Avci, Istanbul (TR); Serdar Soylemez, Istanbul (TR); Fikret Koc, Istanbul (TR); Nadin Narsisoglu, Istanbul (TR)

(21) Appl. No.: 13/141,939

(22) PCT Filed: Dec. 30, 2008
(86) PCT No.: PCT/IB2008/055572

§ 371 Date: Jul. 1, 2011

(51) Int. Cl. A61K 9/28; A61K 31/546; A61P 43/00; A61K 31/41

(52) U.S. Cl. 424/465; 514/382; 514/222.5; 424/474

(57) ABSTRACT

This invention is related to pharmaceutical compositions, which have good flowability properties, comprising olmesartan medoxomil or combination of olmesartan medoxomil and hydrochlorothiazide and a lubricant or mixtures of lubricants and a pharmaceutically acceptable excipient or excipients.

The present invention is related to pharmaceutical compositions, which have good flowability properties, comprising olmesartan medoxomil or combination of olmesartan medoxomil and hydrochlorothiazide and a lubricant or mixtures of lubricants and a pharmaceutically acceptable excipient or excipients.

Degradations of products in pharmaceuticals are important during drug development, since such degradation shall jeopardise efficacy and safety of the pharmaceuticals. Therefore if degradations could be minimum level, saftey will be better. Additionally, if impurities arising from degradation are minimum level, shelf life of product is extended and it means that pecuniary advantage to the manufacturers and consumers along with parts of supply chain.

Olmesartan medoxomil impurity is olmesartan which is called as OL (olmesartan) in an article (Journal of Pharmaceutical and Biomedical Analysis,47 (2008) 553-559, “Identification of a degradation product in stressed tablets of olmesartan medoxomil by the complementary use of HPLC hyphenated techniques”, Murakami et al).

Olmesartan medoxomil is described in EP 0503785 patent.

In Benicar® and Benicar HCT® tablets, which are products of Daiichi Sankyo, magnesium stearate is used as a excipient. EP1336407 patent discloses magnesium stearate, calcium stearate, stearic acid etc. are used as a lubricant. However this patent does not disclose effects of lubricants on reducing or preventing impurities arising from degradation.

Additionally, EP1336407 patent does not disclose which lubricant is more suitable to obtain eligible impurity results. Some of the lubricants are more suitable than other ones to reduce and to prevent impurities in pharmaceutical formulations including olmesartan c. Every lubricant does not have the character to prevent or to reduce impurities in same level.

WO 20071128478 discloses using of stearic acid to achieve stabilization. However stearic acid has bad flowability properties and brings about many problems in industrial scale.

However as a lubricant magnesium stearate is not duly lubricant to prevent impurities since in comparison it is invented that some lubricants entail eligible results rather than magnesium stearate. As a lubricant stearic acid prevents olmesartan acid impurity rather than magnesium stearate. But stearic acid does not provide good flowability as much as magnesium stearate.

It is discovered that some lubricants, have improving effect on stability of olmesartan medoxomil and combination of olmesartan modexomil and hydrochlorothiazide. On the other hand, all of the lubricants do not provide same flowabiity properties to the pharmaceutical powders and lubricants should provide both preventing impurities and providing good flowability. It is also discovered that some lubricants have good flowability properties along with superior effect on stability.

According to this invention lubricants are selected from group of calcium stearate, sodium stearyl fumarate and zinc or mixtures thereof.

According to this invention tablet core of pharmaceutical composition may include other excipients, but are not limited to, fillers such as lactose monohydrate and microcrystalline cellulose, disintegrant such as hydroxypropyl methyl cellulose, binder such as polyvinylpyrrolidone, adherent such as colloidal silica. Coating agent of tablet core may be Opadry® II Pink.

The pharmaceutical composition comprising from 65% to 85% filler, from 8% to 20 active pharmaceutical ingredient or active pharmaceutical ingredients, from 0.2% to 0.5% adherent, from 1% to 5% lubricant, from 1% to 3% binder, from 4% to 7% disintegrant by total weight of the tablet core.

Pharmaceutical composition is preferably oral dosage form. Oral dosage form is preferably tablet. To prepare tablets conventional wet granulation methods may be applied. In wet granulation isopropyl alcohol, but not limited, could be used as wetting agent.

### EXAMPLE 1

TABLE 1
Olmesartan Medoxomil Formulation

| Ingredients | Amount (mg) |
|---|---|
| Olmesartan Medoxomil | 40 |
| Lactose Monohydrate | 242 |
| Povidone K 30 | 6 |
| Hydroxpropylcellulose | 24 |
| Microcrystalline Cellulose | 80 |
| Colloidal Silica | 1 |
| Calcium Stearate | 4 |

### EXAMPLE 2

Five tablet formulations below are prepared with lubricants respectively magnesium stearate,calcium stearate,zinc,sodium stearyl fumarate and stearic acid.

TABLE 2
Tablet Formulation With Magnesium Stearate

| Ingredients | Amount (mg) |
|---|---|
| Olmesartan Medoxomil | 40 |
| Hydrochlorothiazide | 25 |
| Lactose Monohydrate | 255 |
| Povidone K 30 | 8 |
| Hydroxypropylcellulose | 26 |
| Microcrystalline Cellulose | 61 |
| Colloidal Silica | 1 |
| Magnesium Stearate | 4 |

TABLE 3
Tablet Formulation With Calcium Stearate

| Ingredients | Amount (mg) |
|---|---|
| Olmesartan Medoxomil | 40 |
| Hydrochlorothiazide | 25 |
| Lactose Monohydrate | 255 |
| Povidone K 30 | 8 |
| Hydroxypropylcellulose | 26 |
| Microcrystalline Cellulose | 61 |
| Colloidal Silica | 1 |
| Calcium Stearate | 4 |

TABLE 4
Tablet Formulation With Zinc

| Ingredients | Amount (mg) |
|---|---|
| Olmesartan Medoxomil | 40 |
| Hydrochlorothiazide | 25 |
| Lactose Monohydrate | 255 |
| Povidone K 30 | 8 |
| Hydroxypropylcellulose | 26 |
| Microcrystalline Cellulose | 61 |
| Colloidal Silica | 1 |
| Zinc | 4 |

TABLE 5
Tablet Formulation With Sodium Stearyl Fumarate

| Ingredients | Amount (mg) |
|---|---|
| Olmesartan Medoxomil | 40 |
| Hydrochlorothiazide | 25 |
| Lactose Monohydrate | 255 |
| Povidone K 30 | 8 |
| Hydroxypropylcellulose | 26 |
| Microcrystalline Cellulose | 61 |
| Colloidal Silica | 1 |
| Sodium Stearyl Fumarate | 4 |

TABLE 6
Tablet Formulation With Stearic Acid

| Ingredients | Amount (mg) |
|---|---|
| Olmesartan Medoxomil | 40 |
| Hydrochlorothiazide | 25 |
| Lactose Monohydrate | 255 |
| Povidone K 30 | 8 |
| Hydroxypropylcellulose | 26 |
| Microcrystalline Cellulose | 61 |
| Colloidal Silica | 1 |
| Stearic Acid | 4 |

Prepared tablets are stored at 40° C. and 75% relative humidity during one week. Impurity results are below.

TABLE 7
Comparison of Stability and Flowability
Olmesartan Acid Impurity (40° C. and 75% Relative Humidity)

| | | | | Sodium | |
|---|---|---|---|---|---|
| | Magnesium | Calcium | | Stearyl | Stearic |
| Lubricant | Stearate | Stearate | Zinc | Fumarate | Acid |
| Starting | 0.2 | 0.2 | 0.2 | 0.2 | 0.2 |
| Amount | | | | | |
| 1 Week | 0.52 | 0.25 | 0.24 | 0.24 | 0.24 |
| Properties | Stability | Stability | Stability | Stability | Stability |
| | result is | result is | result is | result is | result is |
| | not | eligible | eligible | eligible | eligible but |
| | eligible | and has | and has | and has | has not |
| | but has | flowability | flowability | flowability | flowability |
| | flowability | in in- | in in- | in in- | in in- |
| | in in- | dustrial | dustrial | dustrial | dustrial |
| | dustrial | scale | scale | scale | scale |
| | scale | | | | |

1. A pharmaceutical composition, having improved stability with low impurities and eligible flowability, comprising;
Olmesartan medoxomil or
Combination of olmesartan medoxomil and hydrochlorothiazide and
A lubricant or lubricants and
A pharmaceutically acceptable excipient or excipients and
A coating agent or coating agents of tablet core characterized in that lubricant is selected from group of calcium stearate, zinc and sodium stearyl fumarate or mixtures thereof.

2. According to claim 1, pharmaceutical composition comprising from 65% to 85% filler, from 8% to 20% active pharmaceutical ingredient or combination of active pharmaceutical ingredients, from 0.2% to 0.5% adherent, from 1% to 5% lubricant, from 1% to 3% binder, from 4% to 7% disintegrant by total weight of the tablet core.

3. According to claim 2, filler is lactose monohydrate or microsrystalline cellulose or mixtures thereof

4. According to claim 2, adherent is colloidal silica

5. According to claim 2, binder is povidone K 30

6. According to claim 2, disintegrant is hyroxypropylcellulose

7. According to claim 1, coating agent of tablet core is Opadry® II Pink
